(19) United States
(12) Patent Application Publication
(10) Pub. No.: US 2026/0266754 A1
(43) Pub. Date: Sep. 10, 2026

(54) SENSOR ELEMENT, GAS SENSOR, AND SENSOR ELEMENT MANUFACTURING METHOD

(71) Applicant: Niterra Co., Ltd., Nagoya-shi, Aichi (JP)

(72) Inventors: Kazuma ITO, Nagoya-shi (JP); Wakako NANYA, Nagoya-shi (JP)

(21) Appl. No.: 19/166,551

(22) PCT Filed: Dec. 25, 2023
(86) PCT No.: PCT/JP2023/046425

§ 371 Date: Sep. 18, 2025

(30) Foreign Application Priority Data
Mar. 20, 2023 (JP) 2023-044010

(51) Int. Cl. G01N 27/30; G01N 27/333; G01N 27/407; G01N 27/409; G01N 33/00

(52) U.S. Cl. CPC G01N 27/304; G01N 27/333; G01N 27/4075; G01N 27/409; G01N 33/0027

(57) ABSTRACT

A sensor element for detecting a particular gas present in a measurement object gas, the sensor element including: a first electrode and a second electrode disposed on a solid electrolyte layer; and at least one porous layer which intervenes between the outside and one of the first and second electrodes such that the measurement object gas introduced from the outside to the one electrode passes through the porous layers, wherein the at least one porous layer is a catalyst layer supporting particles of one or more noble metals selected from the group consisting of Pt, Pd, and Rh, and, in an X-ray absorption fine structure analysis, the absorption intensity Ip which is attributed to the noble metal particles supported on the catalyst layer and which is determined at an absorption edge thereof lies between the absorption intensity Im which is attributed to the noble metal particles in the form of elemental metal and which is determined at an absorption edge thereof, and the absorption intensity Io which is attributed to the noble metal particles in the form of oxide which is determined at an absorption edge thereof.

### TECHNICAL FIELD

The present invention relates to a sensor element suitably employed in a gas sensor for determining the particular gas concentration of a combustion gas or an exhaust gas, for example, a gas discharged from a combustor, an internal combustion engine, or the like; to a gas sensor; and to a method for manufacturing the sensor element.

### BACKGROUND ART

As a gas sensor which can determine the specific component concentration of a measurement object gas, such as an exhaust gas discharged from an internal combustion engine such as an automobile, there has been known a cell structure in which a pair of electrodes are formed on the surfaces of an oxygen ion-conductive solid electrolyte (e.g., zirconia) layer. The electrodes are formed by applying a paste containing noble metal (e.g., Pt) particles through screen printing, and firing the applied paste.

Also, in order to enhance the degree of combusting unburnt gas contained in a measurement object gas, there has been known a technique of providing a catalyst layer which covers a detection electrode on the measurement side (Patent Literature 1). The catalyst layer is formed of a porous layer (e.g., an alumina or a titania layer) onto which noble metal particles are deposited and can combust an unburnt gas (including H₂, NOx, or HC) present in the measurement object gas before it reaches the detection electrode, to thereby prevent impedance on the measurement and enhance detection accuracy.

### CITATION LIST

### Patent Literatures

Patent Literature 1: JP2009-186458A

### SUMMARY OF INVENTION

### Technical Problem

Meanwhile, the degree of combusting an unburnt gas (i.e., catalytic activity) increases, as the amount of noble metal particles supported on the catalyst layer increases. However, when the gas is combusted on the catalyst layer, the response speed of a sensor tends to decrease. Conceivably, a drop in sensor response speed stems from the change in characteristics of the measurement object gas (i.e., adsorption and release of oxygen caused by noble metal particles on the catalyst layer).

In view of the foregoing, an object of the present invention is to provide a sensor element that can provide an enhanced detection accuracy by achieving suitable combustion of an unburnt gas present in a measurement object gas and suppress a drop in sensor response speed. Another object is to provide a gas sensor and a method of manufacturing a sensor element in relation thereto.

### Solution to Problem

In order to attain the aforementioned objects, the present invention provides a sensor element for detecting a particular gas present in a measurement object gas, the sensor element having a first electrode disposed on a solid electrolyte layer; a second electrode serving as a counter electrode to the first electrode and disposed on the solid electrolyte layer; and at least one or more porous layers which intervene between the outside and one of the first and second electrodes such that the measurement object gas introduced from the outside to the one electrode passes through the porous layers. A characteristic feature of the sensor element resides in that at least one of the porous layers is a catalyst layer supporting particles of one or more noble metals selected from the group consisting of Pt, Pd, and Rh. Another characteristic feature resides in that, in an X-ray absorption fine structure analysis, an absorption intensity Ip which is attributed to the noble metal particles supported on the catalyst layer and which is determined at an absorption edge thereof lies between an absorption intensity Im which is attributed to the noble metal particles in the form of elemental metal and which is determined at an absorption edge thereof, and an absorption intensity Io which is attributed to the noble metal particles in the form of oxide and which is determined at an absorption edge thereof.

According to the sensor element, the relation: Im<Ip<Io is satisfied. In an X-ray absorption fine structure (XAFS) analysis of the noble metal particles present in the catalyst layer, the electron state assumes a state between that of the elemental metal and that of the metal oxide. Thus, a compromise of two advantageous features can be attained. As a result, an unburnt gas present in the measurement object gas can be suitably combusted to thereby enhance detection accuracy and suppress a drop in response speed of a sensor.

In the sensor element of the present invention, when the noble metal particles include Pt, the Ip of Pt may be 2 to 20%, wherein the Im of Pt is 0%, and the Io of Pt is 100%.

According to the sensor element, the electron state of the noble metal particles present in the catalyst layer can consistently assume a state between that of the elemental metal and that of the metal oxide.

The present invention provides a gas sensor having a sensor element, and a metallic shell holding the sensor element, characterized in that the sensor element is a sensor element as recited in the above.

The present invention provides a method of manufacturing a sensor element for detecting a particular gas present in a measurement object gas, the sensor element having a first electrode disposed on a solid electrolyte layer; a second electrode serving as a counter electrode to the first electrode and disposed on the solid electrolyte layer; and at least one or more porous layers which intervene between the outside and one of the first and second electrodes such that the measurement object gas introduced from the outside to the one electrode passes through the porous layers.

In the sensor element manufacturing method, at least one of the porous layers is a catalyst layer supporting particles of one or more noble metals selected from the group consisting of Pt, Pd, and Rh. A characteristic feature of the sensor element manufacturing method resides in that the method includes a permeation step of causing a noble metal salt solution containing ions or particles of the noble metal to permeate the porous layer to serve as the catalyst layer; and a heat treatment step of conducting a heat treatment of the porous layer which the noble metal salt solution has permeated in an inert atmosphere or a substantially inert atmosphere containing oxygen in an amount of 2,000 mass ppm or less, at a temperature equal to or higher than the thermal decomposition temperature of a component of the noble metal salt solution.

### Advantageous Effect of Invention

According to the present invention, there can be produced a sensor element that can provide an enhanced detection accuracy by achieving suitable combustion an unburnt gas present in a measurement object gas and suppress a drop in response speed of a sensor.

### BRIEF DESCRIPTION OF DRAWINGS

FIG. 1 Sectional view showing a gas sensor 200 which is one embodiment of the present invention.

FIG. 2 Exploded perspective view of a sensor element 10.

FIG. 3 Sectional view of the sensor element 10.

FIG. 4 A graph schematically showing the relationship between catalytic activity and sensor response speed.

FIG. 5 A graph showing the relationship between VR values in H₂-rich atmosphere and absorption intensity of Pt particles present in the catalyst layer.

FIG. 6 A graph showing the relationship between VR values in CO-rich atmosphere and absorption intensity of Pt particles present in the catalyst layer.

FIG. 7 A graph showing the difference (subtraction) between a VR value in H₂-rich atmosphere shown in FIG. 5 and a VR value in CO-rich atmosphere shown in FIG. 6, at any absorption intensity Xμ.

FIG. 8 An XAFS spectrum of Pt particles which assume a state between a metal state (PtM) and an oxide (PtO₂).

FIG. 9 An enlarged portion of the peak area in the spectrum of FIG. 8.

FIG. 10 A graph showing the relationship between the oxygen concentration of the atmosphere in which a noble metal salt solution is subjected to heat treatment and the absorption intensity attributed to Pt particles.

### DESCRIPTION OF EMBODIMENT

An embodiment of the present invention will now be described.

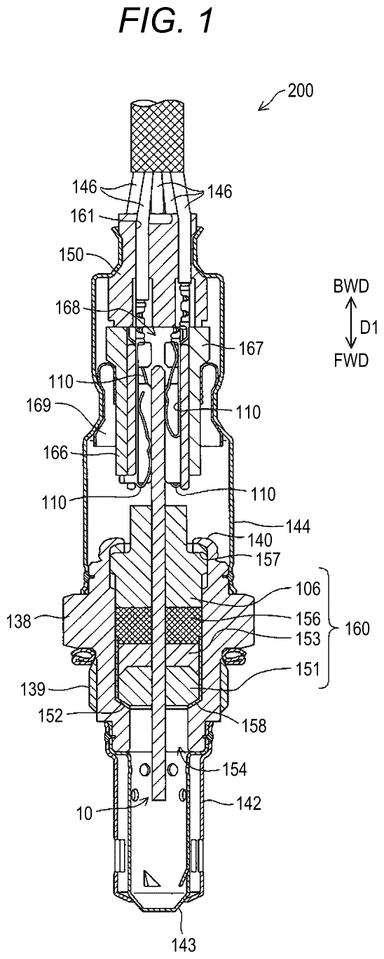

FIG. 1 is a sectional view showing a gas sensor 200 which is one embodiment of the present invention. This gas sensor 200 is fixed to an exhaust pipe of an unillustrated internal combustion engine and measures the concentration of oxygen which is a measurement object gas. FIG. 1 shows a cross section of the gas sensor 200 parallel to a longitudinal direction D1. In the following description, the downward direction (lower side) in FIG. 1 will be referred to as the forward side (FWD) of the gas sensor 200, and the upward direction (upper side) in FIG. 1 will be referred to as the backward side (BWD) of the gas sensor 200.

The gas sensor 200 includes a tubular metallic shell 138, a plate-shaped sensor element 10 extending in the longitudinal direction D1, a tubular ceramic sleeve 106 which surrounds the sensor element 10, an insulating contact member 166, and six connection terminals 110 (in FIG. 1, four connection terminals are shown). A screw portion 139 for fixation to the exhaust pipe is formed on the outer surface of the metallic shell 138.

The ceramic sleeve 106 holds the sensor element 10 therein such that the forward end of the sensor element 10 is located externally on the forward side (FWD) of the ceramic sleeve 106, and a portion of the sensor element 10 on the backward side is located externally on the backward side (BWD) of the ceramic sleeve 106.

The insulating contact member 166 has a contact insertion hole 168 which penetrates the insulating contact member 166 in the longitudinal direction D1. The insulating contact member 166 is disposed such that the inner wall surface of the contact insertion hole 168 surrounds the circumference of a backward end portion of the sensor element 10. Each connection terminal 110 is disposed between the sensor element 10 and the insulating contact member 166.

The metallic shell 138 has a generally tubular shape and a through hole 154 which penetrates the metallic shell 138 in the axial direction and a ledge portion 152 projecting inward in the radial direction of the through hole 154. The metallic shell 138 holds the sensor element 10 in the through hole 154 such that the forward end of the sensor element 10 is located externally on the forward side (FWD) of the through hole 154, and a portion of the sensor element 10 on the backward side is located externally on the backward side (BWD) of the through hole 154. The ledge portion 152 has a taper surface which inclines in relation to a plane perpendicular to the longitudinal direction D1.

This taper surface is formed such that the diameter of the ledge portion 152 on the forward side (FWD) becomes smaller than the diameter of the ledge portion 152 on the backward side (BWD).

Within the through hole 154 of the metallic shell 138, a ceramic holder 151, powder-filled layers 153 and 156 (hereafter referred to also as talc rings 153 and 156) and the ceramic sleeve 106 are stacked in this order from the forward side (FWD) toward the backward side (BWD). The ceramic holder 151, the talc rings 153 and 156, and the ceramic sleeve 106 will be collectively referred to also as a holding portion 160.

The holding portion 160 holds the sensor element 10 within the holding portion 160 such that the forward end of the sensor element 10 is located externally on the forward side (FWD) of the holding portion 160, and a portion of the sensor element 10 on the backward side is located externally on the backward side (BWD) of the holding portion 160. Notably, the sensor element 10 is held by the holding portion 160.

A crimp packing 157 is disposed between the ceramic sleeve 106 and a backward end portion 140 of the metallic shell 138. A metal holder 158 for holding the talc ring 153 and the ceramic holder 151 and for maintaining airtightness is disposed between the ceramic holder 151 and the ledge portion 152 of the metallic shell 138.

Notably, the backward end portion 140 of the metallic shell 138 is crimped to press the ceramic sleeve 106 toward the forward side via the crimp packing 157.

Also, as shown in FIG. 1, an outer protector 142 and an inner protector 143 are attached to the outer circumference of a portion of the metallic shell 138 on the forward side (FWD) by means of welding or the like. The two (double) protectors 142 and 143 are formed of a metal (e.g., stainless steel), each have a plurality of gas introduction holes, and cover a projecting portion of the sensor element 10.

An outer tube 144 is fixed to the outer circumference of a portion of the metallic shell 138 on the backward side. A grommet 150 is disposed in an opening of the outer tube 144 on the backward side (BWD). The grommet 150 has lead insertion holes 161. Six lead wires 146 are inserted into the lead wire insertion holes 161 (only five lead wires 146 are shown in the drawing). These leads 146 are electrically connected to electrode pads (not shown) provided on the outer surface of a portion of the sensor element 10 on the backward side.

Also, the insulating contact member 166 is disposed around a portion of the sensor element 10 on the backward side (BWD), the portion projecting from a backward end portion 140 of the metallic shell 138. This insulating contact member 166 is disposed around electrode pads (not shown) formed on the surface of the portion of the sensor element 10 on the backward side. The insulating contact member 166 has a tubular shape and has a contact insertion hole 168 which penetrates the insulating contact member 166 in the longitudinal direction D1. The insulating contact member 166 has a flange portion 167 projecting outward in the radial direction from the outer surface of a portion of the insulating contact member 166 on the backward side.

A holding member 169 is inserted between the insulating contact member 166 and the outer tube 144. The holding member 169 is in contact with the outer tube 144 and the flange portion 167, whereby the insulating contact member 166 is disposed inside the outer tube 144.

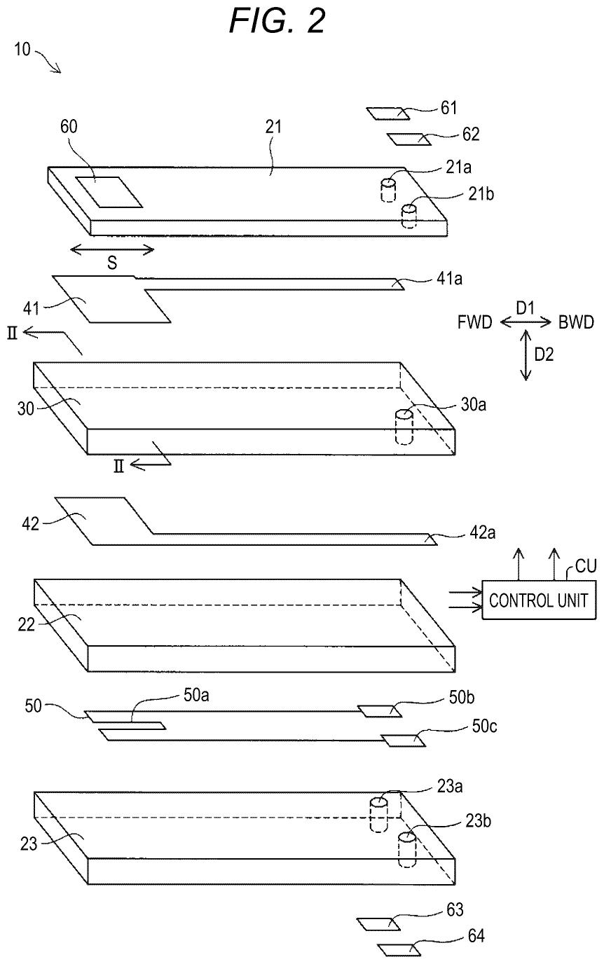

FIG. 2 is an exploded perspective view of the sensor element 10. In FIG. 2, the leftward direction corresponds to the forward direction (forward side) FWD of the sensor element 10, and the rightward direction corresponds to the backward direction (backward side) BWD of the sensor element 10. The sensor element 10 includes an insulating layer 21, a solid electrolyte layer 30, an insulating layer 22, an insulating layer 23, and porous layers 80 and 81. The insulating layer 21, the solid electrolyte layer 30, the insulating layer 22, and the insulating layer 23 are stacked along a stacking direction D2 perpendicular to the longitudinal direction D1. Notably, in FIG. 2, the porous layers 80 and 81 are not shown, and the insulating layer 21, the solid electrolyte layer 30, the insulating layer 22, and the insulating layer 23 are shown.

In the present embodiment, the solid electrolyte layer 30 is formed of mainly zirconia having oxygen ion conductivity, and yttria or calcia is added as a stabilizer.

The insulating layers 21, 22, and 23 are formed of mainly alumina. Each of the solid electrolyte layer 30 and the insulating layers 21, 22, and 23 is formed by using a sheet of a raw material (for example, a sheet of a ceramic such as zirconia or alumina).

A object-gas-side electrode 41 is disposed between the solid electrolyte layer 30 and the insulating layer 21, and a reference gas electrode 42 is disposed between the solid electrolyte layer 30 and the insulating layer 22. A detection lead portion 41a extends from the object-gas-side electrode 41 in the backward direction (toward the backward side) BWD, and a detection lead portion 42a extends from the reference gas electrode 42 in the backward direction (toward the backward side) BWD. Notably, the object-gas-side electrode 41 and the reference gas electrode 42 are formed by using, for example, platinum, rhodium, lead, or the like.

The object-gas-side electrode 41 and the reference gas electrode 42 respectively correspond to “the first electrode” and “the second electrode” in the claims, and the first electrode corresponds to “one electrode” in the claims.

The oxygen concentration of the exhaust gas is detected by an oxygen concentration cell. The oxygen concentration cell is composed of the solid electrolyte layer 30, the object-gas-side electrode 41, and the reference gas electrode 42. The detection lead portion 41a of the object-gas-side electrode 41 is electrically connected to an electrode terminal 61 via a through hole 21a formed in the insulating layer 21. The detection lead 42a of the reference gas electrode 42 is electrically connected to an electrode terminal 62 via a through hole 30a formed in the solid electrolyte layer 30 and a through hole 21b formed in the insulating layer 21.

A porous protection layer 60 formed of alumina or the like is provided in a portion of the insulating layer 21 on the forward side. The porous protection layer 60 is a porous layer provided so as to diffuse the gas (measurement object gas) that enters the object-gas-side electrode 41.

A heater 50 extending along the longitudinal direction D1 is embedded between the insulating layer 22 and the insulating layer 23. The heater 50 is used to heat the sensor element 10 to a predetermined activation temperature so as to increase the oxygen ion conductivity of the solid electrolyte layer, thereby stabilizing operation of the gas sensor 200. The heater 50 is a heat-generating resistor formed of a conductor such as tungsten, and generates heat when electric power is supplied thereto. Notably, the heater 50 is sandwiched between the insulating layer 22 and the insulating layer 23.

The heater 50 has a heat generation portion 50a and electrode terminals 50b and 50c. The heat generation portion 50a is located on the forward side. The heat generation portion 50a includes a heating wire which is meanderingly disposed and generates heat upon energization. The electrode terminals 50b and 50c of the heater 50 are electrically connected to electrode terminals 63 and 64, respectively, through through holes 23a and 23b formed in the insulating layer 23. Notably, the electrode terminals 61 to 64 can be formed by using, for example, platinum, rhodium, lead, or the like.

Notably, in FIG. 2, a control unit CU for the gas sensor 200 (the sensor element 10) is also shown. The heater 50 and the electrode terminals 61 to 64 are connected to the control unit CU via the connection terminals 110 and the lead wires 146 shown in FIG. 1. The control unit CU supplies electric power to the heater 50. In addition, the control unit CU controls the gas sensor 200 (the sensor element 10) by sending signals to the electrode terminals 61 to 64 and receiving signals from the electrode terminals 61 to 64. Notably, the control unit CU may be formed by using a computer having a CPU and a memory.

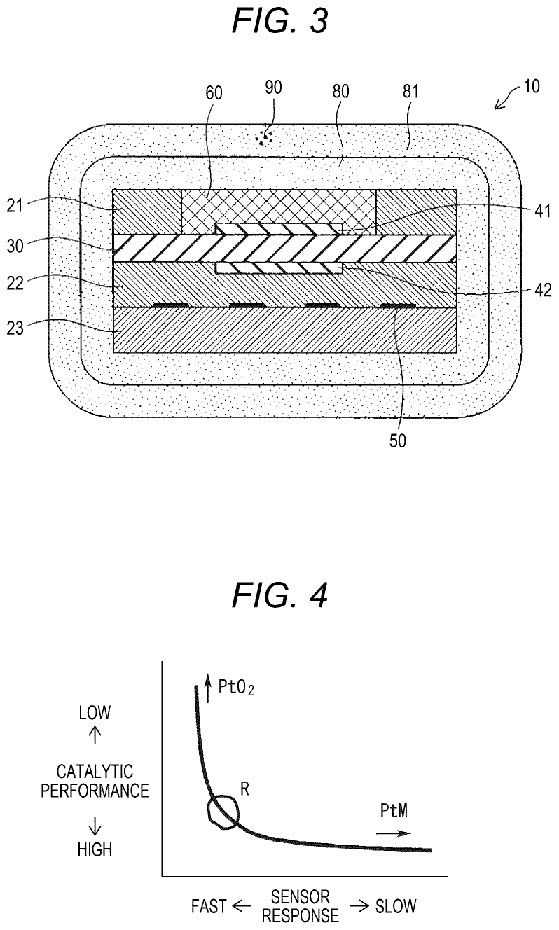

FIG. 3 is a sectional view of the sensor element 10. This sectional view shows a cross section parallel to the stacking direction D2, and a sectional view along a line II-II shown in FIG. 2.

In FIG. 3, the porous layers 80 and 81 whose illustration is omitted in FIG. 2 is shown. In the present embodiment, the porous layers 80 and 81 cover a region S which extends from the forward end of the sensor element 10 to the backward end of the porous protection layer 60. Notably, the porous layer 80 is in contact with the sensor element 10 and covers the sensor element 10, and the porous layer 81 covers the outer surface of the porous layer 80.

The porous layers 80, 81 prevent cracking of the body of the sensor element 10, when water drops are attached to the sensor element 10. In addition, the outer porous layer 81 serves as a catalyst layer supporting one or more species of the noble metal particles 90, the metal being selected from the group consisting of Pt, Pd, and Rh.

The porous layers 80, 81 are mainly formed ceramic particles (alumina particles and spinel particles). Through firing, the ceramic particles are bound together via voids.

The porous layer 81 corresponds to the “catalyst layer” as claimed in the claims. Notably, the relevant catalyst may be supported not only on the outermost layer (porous layer 81), but also on the inner layer (porous layer 80).

The noble metal particles 90 supported on the porous layer 81 serve as a catalyst that can enhance the degree of combustion of the unburned gas. That is, the noble metal particles 90 help combustion of unburned gases (e.g., H₂, NOx, and HC) present in the measurement object gas (i.e., exhaust gas) before reaching the object-gas-side electrode 41, to thereby prevent intervention to the measurement, whereby detection accuracy is enhanced.

Meanwhile, the degree of combustion (i.e., catalytic activity) of the unburned gas varies depending on the conditions (e.g., oxidation state and particle size) and amount of the noble metal particles 90 supported on the catalyst layer. For example, as the amount of the noble metal particles 90 increases, the degree of combustion (i.e., catalytic activity) of the unburned gas increases, but the response speed of the sensor tends to decrease due to combustion of the gas on the catalyst layer.

FIG. 4 is a graph schematically showing the relationship between catalytic activity and sensor response speed. A trade-off relationship is established between catalytic activity and sensor response speed. A conceivable reason for a drop in response speed of a sensor when the amount of the noble metal particles 90 supported on the catalyst layer increases is the change in characteristics of the measurement object gas (i.e., adsorption and release of oxygen caused by noble metal particles on the catalyst layer).

Further, according to the studies conducted by the present inventors, when merely a low catalytic activity is achieved even by use of the same amount of the noble metal particles 90, the noble metal particles 90 (Pt in FIG. 4) are likely to assume an oxide form (PtO₂ in FIG. 4). In contrast, when the response speed of a sensor is low, the noble metal particles 90 (Pt in FIG. 4) are likely to assume a metal oxide state (PtM in FIG. 4).

Therefore, if the state of the noble metal particles 90 in region R in FIG. 4, where both catalytic activity and sensor response speed are suitably achieved, can be specifically determined, possibly, an unburned gas in the measurement object gas would be suitably combusted to thereby enhance detection accuracy and suppress a drop in sensor response speed.

Under such circumstances, the present inventors have experimentally determined an index for specifying the state of the noble metal particles 90 in region R in FIG. 4.

Specifically, the noble metal particles 90 supported on a catalyst carrier was prepared through the following procedure. Firstly, a noble metal salt solution containing Pt ions (specifically, a solution of dinitrodiammineplatinum (Pt) nitrate salt) is caused to permeate the porous layer 81. Then, the entirety of the sensor element 10 including the porous layer 81 was subjected to a heat treatment in a specific atmosphere at a temperature equal to or higher than a temperature where a component of the noble metal salt solution is thermally decomposed, to thereby yield the noble metal particles 90.

By changing the Pt amount in the noble metal salt solution and the heat treatment atmosphere in a various way, the electron state of the yielded Pt particles was caused to be varied between the metal state (PtM) and the oxide state (PtO₂).

As a result, when the oxygen level of the heat treatment atmosphere is lower, the electron state approached a metal state, and when the oxygen level of the heat treatment atmosphere is higher, the electron state approached a metal state. The latter behavior is unexpected, and the details will be described later.

The electron state of Pt particles obtained through heat treatment was characterized on the basis of an absorption intensity Xμ (X-ray absorption amount or absorbance) at an absorption edge in an X-ray absorption fine structure (XAFS) analysis.

Notably, a Pt foil assuming a metal state (PtM) was found to exhibit an Xμ of 1.237 (arbitrary unit: a.u), and that assuming an oxide state (PtO₂) was found to exhibit an Xμ of 2.214.

The thus-fabricated sensor element 10 was attached to a gas sensor 200, and the sensor output with respect to a specific test gas atmosphere was measured.

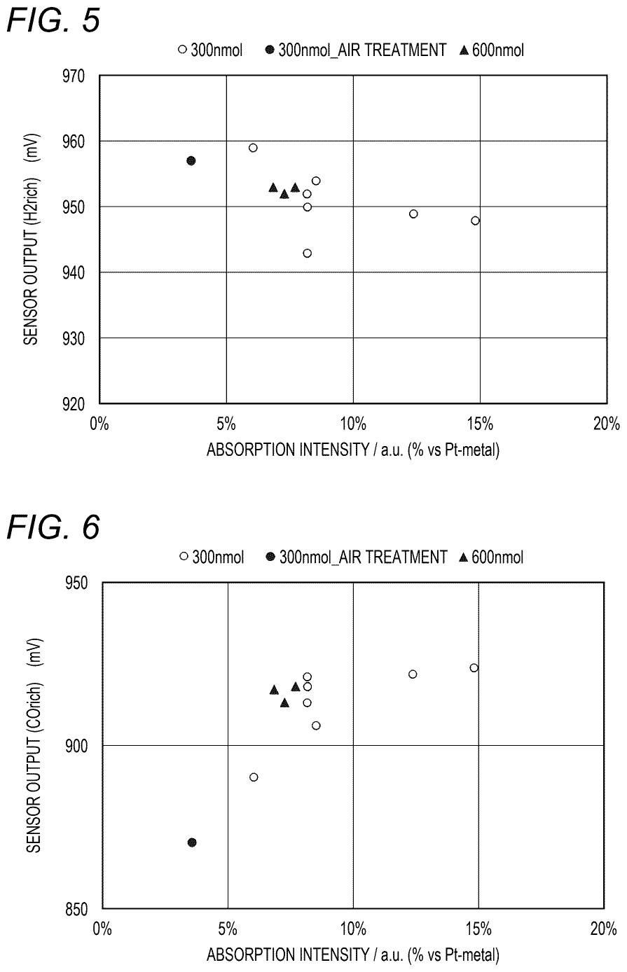

FIGS. 5 and 6 are graphs showing the relationship between VR values in H₂-rich atmosphere and absorption intensity of Pt particles present in the catalyst layer, and the relationship between VR values in CO-rich atmosphere and absorption intensity of Pt particles present in the catalyst layer, respectively. As is clear from FIG. 5, when the absorption intensity Xμ (the left side in FIG. 5) is lower, the electron state of Pt particles approaches a metal state (PtM), and when the absorption intensity Xμ (the right side in FIG. 5) is higher, the electron state of Pt particles approaches an oxide state (PtO₂).

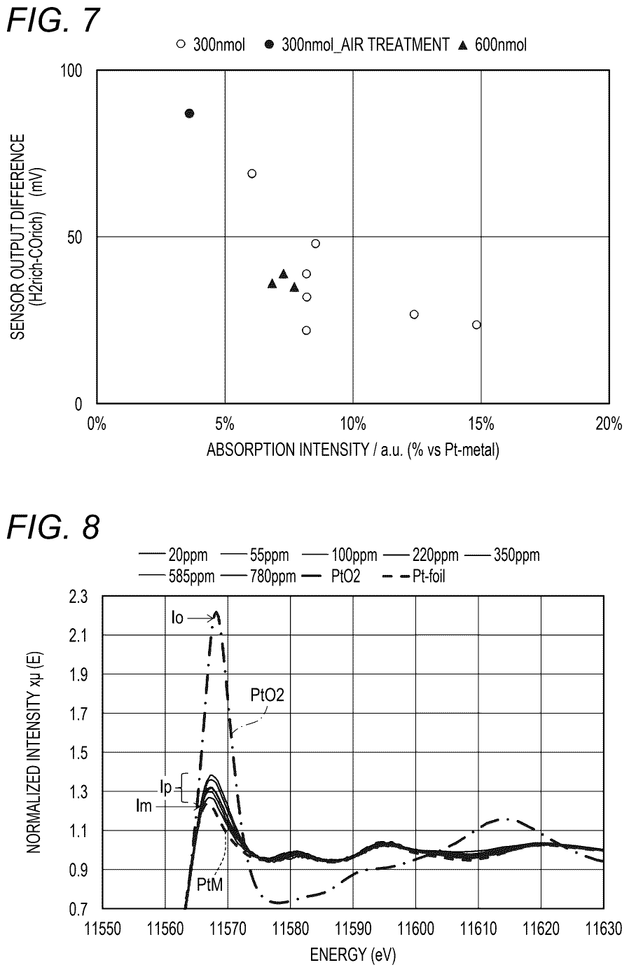

Also, FIG. 7 is a graph showing the difference (subtraction) (VR(H₂—CO)) between a VR value in H₂-rich atmosphere shown in FIG. 5 and a VR value in CO-rich atmosphere shown in FIG. 6, at any absorption intensity Xμ.

When the difference (VR(H₂—CO)) in VR value shown in FIG. 7 is smaller, consistent detection can be achieved in no response to the composition of the measurement object gas; i.e., excellent detection accuracy can be achieved, which is preferred.

Table 1 shows VR(H₂), VR(CO), and difference VR(H₂—CO)) in a metal state (PtM: Pt foil) and in an oxide state (PtO₂).

TABLE 1

| | VR(H2)/mV | VR(CO)/mV | VR(H2 − CO) |
|---|---|---|---|
| PtM | 1041.6 | 1039.5 | 2.1 |
| PtO2 | 956 | 887 | 69 |

As shown in Table 1, in a metal state (PtM), the difference VR is considerably small, and excellent detection accuracy can be achieved. However, as shown in FIG. 4, the response speed of a sensor is low. In contrast, in an oxide state (PtO₂), a large difference VR and an excellent response speed can be achieved, but detection accuracy is not excellent.

As a result, it has found to be suitable that the difference VR value corresponds to a state between a metal state (PtM) and an oxide state (PtO₂).

Notably, the metal state (PtM) was established by setting the atmosphere in which the noble metal salt solution is heat-treated to a reducing atmosphere (H₂), and the oxide state (PtO₂) was established by setting the atmosphere in which the noble metal salt solution is heat-treated to air.

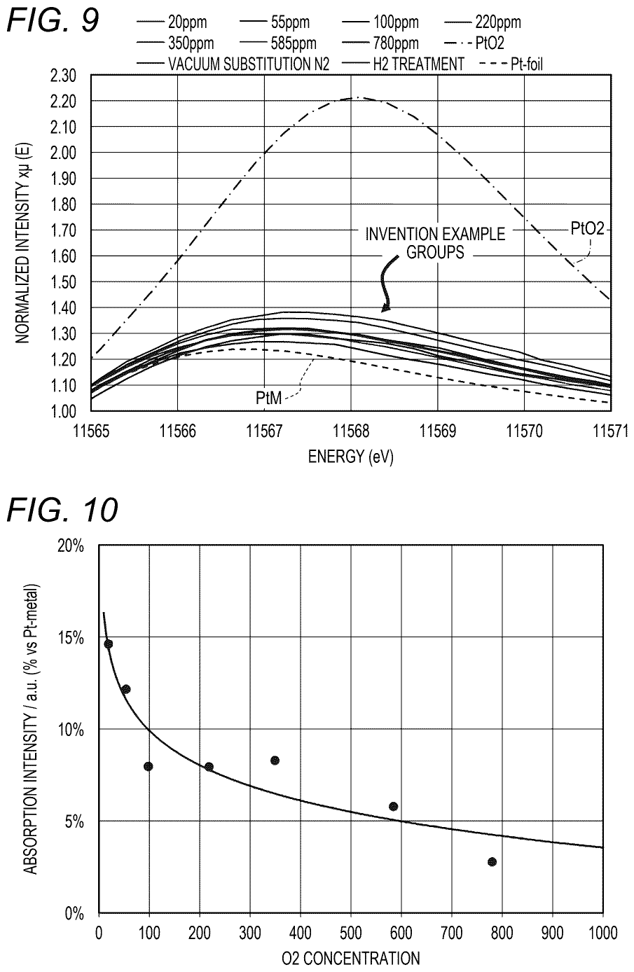

Then, FIGS. 8 and 9 show an XAFS spectrum of Pt particles exhibiting a difference VR value corresponding to a state between a metal state (PtM) and an oxide state (PtO₂) (“the invention example group” in FIG. 9), along with XAFS spectra of Pt particles in a metal state (PtM) and an oxide state (PtO₂). Notably, FIG. 9 is an enlarged portion of the peak area in the spectrum of FIG. 8.

As is clear from FIGS. 8 and 9, in XAFS, the absorption intensity Ip which is attributed to the Pt particles and which is determined at an absorption edge thereof lies between the absorption intensity Im which is attributed to the Pt particles in a metal state (PtM: elemental metal) and which is determined at an absorption edge thereof, and the absorption intensity Io which is attributed to the Pt particles in the form of oxide (PtO₂) and which is determined at an absorption edge.

Thus, the present invention has been accomplished on the basis of the relationship: Im<Ip<Io, which serves as an index for characterizing the state of the noble metal particles 90 in region R in FIG. 4.

When the relationship Im<Ip<Io is satisfied, the noble metal particles 90 present in the catalyst layer assume a state corresponding to region R in FIG. 4; i.e., an electronic state between that of elemental metal and metal oxide. As a result, a compromise of two advantageous features can be attained, whereby an unburnt gas present in the measurement object gas can be suitably combusted to thereby enhance detection accuracy and suppress a drop in response speed of a sensor.

Notably, as mentioned above, the actual measurements of Im and Io of Pt were 1.237 and 2.214, respectively. Ip values of the Example groups shown in FIG. 9 were found to be 1.267 to 1.382.

On the basis of calculation from the data, when the noble metal particles present in the catalyst layer include Pt, with Im and Io of Pt are respectively fixed to 0% and Io 100%, Ip of Pt is preferably 2 to 20%.

For example, in the case where the noble metal particles are in the form of a mixture of Pt and Rh, the concept “the noble metal particles present in the catalyst layer include Pt” encompasses Ip of Pt in the mixture of Pt and Rh of 2 to 20%.

Next, an operational example of the sensor element 10 will be described. Firstly, the control unit CU controls to supply electric power to the heater 50, and the heater 50 heats the object-gas-side electrode 41 and the reference gas electrode 42 to an activation temperature. Once the object-gas-side electrode 41 and the reference gas electrode 42 have been heated to the activation temperature, the control unit CU controls to supply current between the object-gas-side electrode 41 and the reference gas electrode 42. As a result, an amount of oxygen to attain a reference concentration is transferred from the object-gas-side electrode 41 to the reference gas electrode 42.

In response to the oxygen concentration of the measurement object gas (exhaust gas) flown into the object-gas-side electrode 41, the electromotive force between the object-gas-side electrode 41 and the reference gas electrode 42 drastically steeply varies at about the theoretical air fuel ratio (λ=1). As a result, the control unit CU determines whether the measurement object gas (exhaust gas) is in a lean state or a rich state. As used herein, the term “lean state” refers to an atmosphere in which the relative amount of oxygen with respect to λ=1 is larger, and the term “rich state” refers to an atmosphere in which the relative amount of oxygen with respect to λ=1 is small.

Notably, in the present Example, the reference gas is stored in the reference gas electrode 42. However, a space (i.e., an air-introducing hole) may be provided between the reference gas electrode 42 and the insulating layer 22.

Next, the sensor element manufacturing method according to the embodiment of the present invention will be described.

The sensor element manufacturing method according to the embodiment of the present invention includes a permeation step of causing a noble metal salt solution containing ions or particles of the noble metal to permeate the porous layer to serve as the catalyst layer; and a heat treatment step of conducting a heat treatment of the porous layer which the noble metal salt solution has permeated in an inert atmosphere or a substantially inert atmosphere containing oxygen in an amount of 2,000 mass ppm or less, at a temperature equal to or higher than the thermal decomposition temperature of a component of the noble metal salt solution.

According to the aforementioned experimental results, the electron state of the obtained noble metal particles (e.g., Pt particles) is modified to a state between a metal state (PtM) and an oxide state (PtO₂) by the atmosphere of the heat treatment of the porous layer which the noble metal salt solution has been permeated.

Thus, the heat treatment atmosphere is a key factor.

The reasons for tuning the heat treatment atmosphere to an inert atmosphere or a substantially inert atmosphere containing oxygen in an amount of 2,000 mass ppm or less are as follows.

Specifically, as mentioned above, if the heat treatment atmosphere is tuned to a reducing atmosphere (H₂), a noble metal (e.g., Pt) oxide is thoroughly reduced to provide a metal state (PtM). Thus, such a reducing atmosphere is excluded from the inert atmosphere. Examples of the inert atmosphere include rare gas (e.g., Ar) and N₂ gas.

Also, as mentioned above, if the heat treatment atmosphere is tuned to air (oxygen content: about 200,000 mass ppm), a noble metal (e.g., Pt) is thoroughly oxidized. Thus, an inert atmosphere is employed.

However, surprisingly, as shown in FIG. 10, it has been found that, in an inert atmosphere containing oxygen in an amount of 2,000 mass ppm or less, more preferably 1,000 ppm or less, still more preferably 100 ppm or less, the electron state approaches a metal state (PtM) as the oxygen concentration increases.

Since Xμ of the metal state (PtM) is 1.237, an oxygen concentration which provides an Xμ higher than 1.237 is estimated to be about 1,000 mass ppm or higher. Thus, even when the inert atmosphere contains oxygen in an amount of 1,000 mass ppm or less, the electron state of the noble metal particles can be adjusted to a state between a metal state (PtM) and an oxide state (PtO₂).

Notably, in FIG. 10, the lowest level of oxygen concentration is 20 mass ppm, and the highest level is 780 ppm.

Examples of the method of causing the noble metal salt solution to permeate the porous layer 81 include spray coating and impregnation. The temperature of the heat treatment may be tuned to about 900 to about 1100° C.

The present invention is not limited to the aforementioned embodiment. needless to say, the invention also encompasses various modifications and equivalents falling within the sprit and scope of the invention. For example, the present invention may be applied, in a non-limitative way, to oxygen sensors such as a NOx sensor and as a full range air fuel ratio sensor.

Specifically, in the aforementioned Example, the sensor element 10 has a plate shape in which component layers are stacked. However, the sensor element is not limited to a plate-shape sensor element, and may be formed into a cylindrical sensor element. Notably, the term “cylindrical sensor element” refers to a sensor element having a structure having a center heater and a solid electrolyte layer disposed to surround the heater. More specifically, the sensor element refers to a cylindrical sensor element having a heater, a reference gas electrode, a solid electrolyte layer, an object-gas-side electrode, and a porous layer, disposed from the center to the outside toward a radial direction.

Also, in the aforementioned Example, the porous layer supporting the catalyst metal has a bi-layer structure (porous layers 80, 81). However, a mono-layer structure may also be employed. When a mono-layer structure is employed, the size of the sensor element can be reduced. In contrast, the porous layer supporting the catalyst metal may have a structure of 3 or more layers. By increasing the number of porous layers, invasion of water into the sensor element can be securely prevented.

### REFERENCE SIGNS LIST

10 sensor element
30 solid electrolyte layer
41 first electrode, one electrode (object-gas-side electrode)
42 second electrode (reference gas electrode)
80 porous layer
81 porous layer (catalyst layer)
90 noble metal particles
138 metallic shell
200 gas sensor

What is claimed is:

1. A sensor element for detecting a particular gas present in a measurement object gas, the sensor element comprising: a first electrode disposed on a solid electrolyte layer; a second electrode serving as a counter electrode to the first electrode and disposed on the solid electrolyte layer; and at least one porous layer which intervenes between the outside and one of the first and second electrodes such that the measurement object gas introduced from the outside to the one electrode passes through the porous layers,
wherein the at least one porous layer is a catalyst layer supporting particles of one or more noble metals selected from the group consisting of Pt, Pd, and Rh, and,
wherein in an X-ray absorption fine structure analysis, an absorption intensity Ip which is attributed to the noble metal particles supported on the catalyst layer and which is determined at an absorption edge thereof lies between an absorption intensity Im which is attributed to the noble metal particles in the form of elemental metal and which is determined at an absorption edge thereof, and an absorption intensity Io which is attributed to the noble metal particles in the form of oxide and which is determined at an absorption edge thereof.

2. The sensor element according to claim 1, wherein,
when the noble metal particles include Pt, the Ip of Pt is 2 to 20%, the Im of Pt is 0%, and the Io of Pt is 100%.

3. A gas sensor comprising a sensor element, and a metallic shell holding the sensor element,
wherein the sensor element is a sensor element as recited in claim 1.

4. A method of manufacturing a sensor element for detecting a particular gas present in a measurement object gas, the sensor element having a first electrode disposed on a solid electrolyte layer; a second electrode serving as a counter electrode to the first electrode and disposed on the solid electrolyte layer; and at least one porous layer which intervenes between the outside and one of the first and second electrodes such that the measurement object gas introduced from the outside to the one electrode passes through the porous layers,
wherein the at least one porous layer is a catalyst layer supporting particles of one or more noble metals selected from the group consisting of Pt, Pd, and Rh, and
wherein the method comprises;
a permeation step of causing a noble metal salt solution containing ions or particles of the noble metal to permeate the porous layer to serve as the catalyst layer; and
a heat treatment step of conducting a heat treatment of the porous layer which the noble metal salt solution has permeated in an inert atmosphere or a substantially inert atmosphere containing oxygen in an amount of 2,000 mass ppm or less, at a temperature equal to or higher than the thermal decomposition temperature of a component of the noble metal salt solution.
